(19) United States
(12) Patent Application Publication
(10) Pub. No.: US 2010/0203549 A1
(43) Pub. Date: Aug. 12, 2010

(54) CALIBRATED RPMA ASSAY

(75) Inventors: Emanuel F. Petricoin, III, Gainesville, VA (US); Lance A. Liotta, Bethesda, MD (US)

Correspondence Address: THE LAW OFFICE 0F GEOFFREY M. KARNY, 2152 BONAVENTURE DR., VIENNA, VA, 22181, US

(73) Assignee: George Mason Intellectual Properties, Inc

(21) Appl. No.: 12/676,257

(22) PCT Filed: Sep. 5, 2008
(86) PCT No.: PCT/US08/10445

§ 371 Date: Mar. 3, 2010

Related U.S. Application Data
(60) Provisional application No. 60/970,325, filed on Sep. 6, 2007.
(60) Provisional application No. 61/071,324, filed on Apr. 22, 2008.

(51) Int. Cl. G01N 33/53; C08B 5/02

(52) U.S. Cl. 435/7.1; 536/56

(57) ABSTRACT

This invention relates, e.g., to a set of calibrants for determining the amount in a sample of an analyte (e.g., a protein, such as a protein that has been post-translationally modified), comprising a plurality of calibrants, which contain a range of amounts (e.g., defined amounts and/or serial dilutions) of the analyte, spanning the expected amount of the analyte in the sample. In each of the calibrants, a defined amount of the analyte is present in the same suitable, biological diluent (e.g., a cell or tissue lysate, or a bodily fluid). In one embodiment of the invention, the diluent reflects the same or a similar biological milieu (proteins, lipids, serum proteins, serum matrix proteins, etc.) as that in the sample in which the analyte to be measured is present. In embodiments of the invention, a single calibrant (e.g., a cell lysate) may comprise as many as hundreds of analytes, and can be used for the quantification of those hundreds of analytes in a sample. Methods are described for performing an assay (e.g. RPMA analysis), in which the calibrants of a set of calibrants of the invention are immobilized on each of the surfaces to which samples to be analyzed are immobilized, thereby providing an internal calibration curve for quantifying an RPMA assay.

This application claims the benefit of the tiling date of U.S. Provisional Application Ser. No. 60/970,325, filed Sep. 2, 2007 and of U.S. Provisional Application Ser. No. 61/071,324, filed Apr. 22, 2008, the disclosures of which are incorporated by reference herein in their entirety.

### BACKGROUND INFORMATION

Reverse phase protein microarray (RPMA) analysis is a method in which aliquots of samples of, e.g., bodily fluids or lysed tissues are immobilized on a surface, such as a slide, and analytes in the aliquots are probed with a first antibody that is specific for an analyte of interest in the sample and a second, detectably labeled, antibody that is specific for the first antibody, to determine the amounts of the analytes in the samples. The method allows for the determination of analytic concentrations of extremely small quantities of analyte in the samples. See, e.g., Sheehan et al. (2005) Mol Cell Proteomics 4, 346-365; Pawaletz et al. (2001) Oncogene 20, 1981-1989; or Nishizuka et al. (2003) Proc. Natl. Acad. Sci. 100, 14229-14239 for descriptions of RPMA. Currently, RPMA analysis requires the use of colorimetric-based assays using third-generation amplification chemistries (e.g. tyramide precipitation/deposition). This approach provides great analytical sensitivity, which is important for the successful analysis of tissue biopsy specimens in which the cellular content is very low, or body fluid analysis in which only a few microliters of samples are provided (e.g. vitreous fluid sampling). However, because of the very poor dynamic range of colorimetric systems, the ability to determine the concentration of an analyte in an input sample so that antigen-antibody interactions are within the linear dynamic range requires that the sample be printed in a miniature dilution curve, usually a series of 4 to 5 1:2 dilutions. The requirement for a printed dilution curve to insure that a linear dynamic range is captured for an unknown starting concentration of analyte requires high-end sophisticated image processing and bioinformatics (both parametric and non-parametric) to distill the final intensity value or analyte concentration value. Manipulations such as curve fitting, slope finding, factor averaging, “super curve” analysis or non-parametric analysis are generally used to analyze the dilution curve from each sample on the RPMAs. There is a need for a method that does not require that a dilution curve be printed for the RPMA, and that provides a facile and accurate means of intensity calculation and analyte concentration calculation. Such a method would be useful, for example, for the implementation of RPMA assays, including multiplex assays, in the clinic.

### DESCRIPTION OF THE DRAWINGS

FIG. 1 shows a schematic illustration of a method of the invention. FIG. 1A shows a slide on which are immobilized: (a) samples from 5 patients, each sample in triplicate in a two point dilution series. The samples have been incubated with a primary antibody that is specific for one analyte of interest in the sample, and a secondary antibody which is specific for the primary antibody and which is coupled to a fluorophore. The level of fluorescence is indicated by different shades of gray; (b) built-in calibration curves: four copies of a set of calibrants (sometimes referred to herein as a “calibration curve”), which comprises 7 calibrants, each of which has a different amount of the analyte of interest; and (c) built-in independent low and high controls. FIG. 1B shows a plot of the amount of fluorescence of the calibrators in the set of calibrants, fitted as a non-parametric curve fit. The amount of fluorescence of the undiluted samples from one of the patients is compared to the values of the set of calibrants, indicating the amount of the analyte in the patient sample.

### DESCRIPTION OF THE INVENTION

The inventors describe herein a method for quantitating an immunologic assay, in particular an RPMA assay, which does not require that a sample to be analyzed be diluted in a miniature dilution curve to ensure that analytes of interest in the sample are present in an amount that is within the dynamic range of the assay. A method of the invention employs an internal set of calibrants (sometimes referred to herein as a “calibration curve”), which is present on each surface on which aliquots of samples for analysis are immobilized. The set of calibrants comprises calibrants which cover a range of amounts (concentrations) of the analytes that are to be measured in the samples; in each of the calibrants, a defined amount of an analyte to be quantitated is diluted into the same, suitable, biological diluent (e.g., a cell or tissue lysate, or a bodily fluid). In one embodiment of the invention, the diluent reflects the same or a similar biological milieu (proteins, lipids, serum proteins, serum matrix proteins, etc.) as that in the sample in which the analyte to be measured is present. The components of this biological milieu are sometime referred to herein as biological components. For example, for samples that are lysates of cells or tissues, each calibrant can comprise a defined amount of an analyte to be measured, in a background of a lysate of cultured cells. Because an analyte in a calibrant is in the same or a similar biological background as the analyte in the sample, analytes in both the sample and the calibrants will be subject to the same effects (e.g., masking, competition in the assay, etc.) of background proteins, lipids, etc. For samples from bodily fluids, each calibrant can comprise a defined amount of an analyte to be measured, diluted into a comparable bodily fluid which lacks, or contains very low amounts of, the analyte. The presence of a constant amount of biological components of a cell, tissue or bodily fluid in each of the calibrants (as well as in the sample aliquot) ensures that the signal intensity of the analyte in each aliquot in the set of calibrants reflects the amount of the measured analyte and not the amount of other biological components in the sample.

A “calibrant” for an analyte of interest, as used herein, refers to a composition which comprises a defined amount or concentration of the analyte, and which can be used as a comparative reference standard for that analyte. The calibrant may also comprise defined amounts of other analytes, which can be used for quantitating the amount of those analytes in a sample, as well. For example, the calibrant can be a cell lysate, or a diluted cell lysate, which contains many proteins that can serve as reference standards for protein analytes of interest. In one such embodiment, for example, a cell line is treated with calyculin (a serine/threonine phosphatase inhibitor), lysed, and used as a calibrant. This lystate can contain defined amounts of hundreds of proteins, including phosphorylated proteins, and can thus can be queried with different specific (e.g., phospho-specific) antibodies to quantitate the hundreds of different proteins (e.g., phosphoproteins).

By way of example, consider an RPMA assay to determine in a sample (e.g., a bodily fluid or a tissue lysate) the amount of one of the activated (phosphorylated) forms of a protein (isoforms) as listed in Table 3 (e.g., Pyk2 that is phosphorylated at the Y402 site). To prepare a set of calibrants for quantitating the amount of this phosphoprotein isoform, upper and lower calibrants are generated. In the present example, the upper calibrant is a cell lysate which contains relatively high levels of the phosphoprotein isoform of interest. One can prepare such an upper calibrant by, for example, incubating HeLa cells in the presence of Pervanadate. The lower calibrant is a cell lysate in which the protein of interest is not phosphorylated, is phosphorylated to a low degree, or is phosphorylated at a different amino acid residue. One can prepare such a lower calibrant by, for example, lysing approximately the same number of HeLa cells, wherein the HeLa cells have not been incubated with Pervanadate. To generate a set of calibrants having a range of intermediate calibrants for the phosphoprotein isoform of interest, one can mix different ratios of the upper and the lower calibrants, so that the amount of the phosphoprotein isoform of interest in each of the intermediate calibrants falls within a desired range, but the total amount of biological components in each of the calibrants is held constant. If other phosphoprotein isoforms from Table 3 are also to be analyzed along with the first phosphoprotein isoform, the same set of calibrants can be used to quantitate all of these phosphoprotein isoforms. (Phosphoprotein isoforms are sometimes referred to herein as “endpoints.”) Alternatively, one can prepare a set of calibrants by diluting known concentrations of one or more purified or substantially purified analytes of interest (e.g., phosphoprotein isoforms or peptides in which the desired residue is phosphorylated) into a cell or tissue lysate or into a biological fluid, which lacks the analyte(s) of interest. The resulting calibrants will contain a range of amounts of the analyte, but the same total concentration of biological components from the cell, tissue or bodily fluid.

An aliquot of the sample to be analyzed is immobilized on a surface in a confined zone, which can receive an individual reagent treatment. When many aliquots are immobilized on a surface, e.g., in an array, and/or are to be analyzed automatically (e.g., robotically), it is sometimes useful to immobilize the aliquots at defined positions. In the present exemplary assay, the surface is a slide. If other samples are to be analyzed for the same phosphoprotein isoform, aliquots of those samples are also immobilized on the slide (e.g., at defined positions), for example to create an array or microarray of aliquots. Calibrants of the set of calibrants are also immobilized on the slide (e.g., in a linear orientation). A primary antibody which is specific for the phosphoprotein isoform of interest is then contacted with the aliquots on the slide, including the calibrants, under conditions that are effective for the primary antibody to interact (bind) specifically with the phosphoprotein isoform of interest, in those aliquots which contain the phosphoprotein isoform. A detectably labeled secondary antibody, which is specific for the first antibody, is then contacted with the slide, under conditions effective for the secondary antibody to interact (bind) specifically to the first antibody. The amount of signal from the label of the secondary antibody is proportional to the amount of the phosphoprotein isoform in an aliquot of the sample.

If additional phosphoprotein isoforms, such as others listed in Table 3, are to be analyzed, separate slides are prepared, one for each of the phosphoprotein isoforms to be analyzed; and aliquots of the samples, as well as the calibrants of the set of calibrants, are immobilized on each slide. On each slide, a primary antibody specific for the phosphoprotein isoform to be analyzed is contacted with the slide, and the secondary antibody is contacted with the slide, as above.

Note that the calibrants described above comprise all 25 of the phosphoprotein isoforms listed in Table 3. Therefore, the same set of calibrants can be used to quantitate any of these 25 analytes if they are present in a sample. The 25 analytes may be present in the sample in any of a wide range of amounts/concentration, spanning a range of at least two orders of magnitude, so the amount of signal emanating from each analyte in an aliquot of the sample which has been printed on a surface may fall anywhere within this at least two orders of magnitude range. Nevertheless, all 25 of the analytes can be quantitated using the same set of calibrants, provided that the detectable label on the secondary antibody (e.g., a fluorescent label) has a dynamic range of at least two orders of magnitude. A method of the invention, using a set of calibrants of the invention, is particularly useful for performing such multiplex assays.

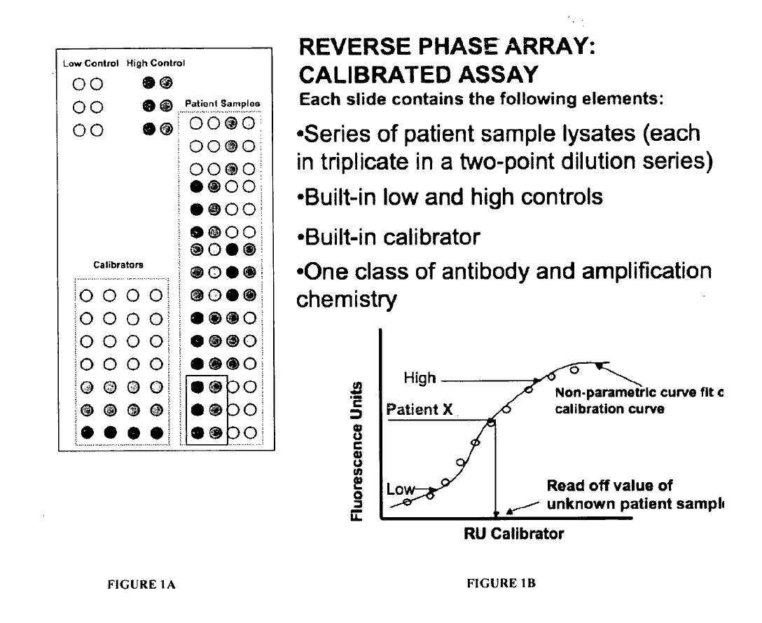

Binding of the secondary, detectably labeled antibody is performed and the signal from this antibody is measured (e.g., recorded) for each of the aliquots on each slide. Thereafter, an investigator can determine the relative or exact amount (concentration) of the phosphoprotein in the sample by extrapolating the signal generated by the phosphoprotein to a non-parametrically or parametrically determined curve fit of the set of calibrants. See FIG. 1 for a diagrammatic representation of such an assay.

Advantages of a method of the invention include that it is simple, fast, reproducible and accurate. In some embodiments, a method of the invention allows an investigator to perform multiplex assays, and to quantify as many as hundreds of different analytes, or more, in a sample with just a few calibrants. Furthermore, because a dilution curve of the sample is not used in a method of the invention, it is not necessary to employ the sophisticated and complicated analysis required to generate a single value from many values from each and every sample (e.g. complex parametric or non-parametric type informatics for intensity value determination in each sample). Rather, the curve fitting techniques are confined to the small number of calibrants in a set of calibrants of the invention. Also, because a dilution curve is not required for each sample, the space to print many samples on each slide greatly increases, which can provide a significant cost savings. Moreover, by eliminating the need to dilute each experimental test sample to ensure that it lies within the linear range of the assay, one can measure many more endpoints/analytes from each lysate. For example, a typical dilution curve used in previous methods contains a series of 1:2 dilutions of any given lysate; the first 1:2 dilution uses up ½ A of the total lysate, effectively cutting in half the number of slides one could print if only the neat spot alone were printed. By eliminating the need to make serial dilutions of the sample, a method of the invention also eliminates the compounding error rates which result from sequential dilution pipetting. This allows for more accurate and precise determinations, and greater robustness through a lowered CV (coefficient of variance) for each analysis.

The present invention relates, e.g., to a set of calibrants for determining the amount in a sample of an analyte (e.g., a protein, such as a protein that has been post-translationally modified), comprising a plurality of calibrants, each of which contains an amount of the analyte (e.g., a defined amount and/or serial dilution) which falls with a range of amounts that span the expected amount of the analyte in the sample. For example, members of a population of subjects might be expected to have between one copy and 100 copies of an analyte of interest; in such a case, the calibrants for that analyte should span the range of one and 100 copies of the analyte. By “span” the range is meant that the calibrants cover the range of one (or fewer) to 100 (or more) copies of the analyte. In each of the calibrants, the analyte is present in the same, suitable, biological diluent. A suitable biological diluent (a diluent comprising biological components, rather than a simple buffer) will vary according to the sample being analyzed. For example, if a sample from a tissue or cell lysate is being analyzed, a biological diluent comprising a cell culture lysate may be used. If a sample from a bodily fluid is being analyzed, the biological diluent may be a comparable bodily fluid. The set of calibrants may be used, e.g., for determining the amount in the sample of at least three analytes; such a set of calibrants comprises a plurality of calibrants, containing a range of amounts of each of the at least three analytes which span the expected amount of each of the analytes in the sample, wherein the total amount of biological components in each of the calibrants is constant.

A set of calibrants is sometimes referred to herein as a “calibration curve.” This usage is distinct from a calibration curve plot, or curve fit, which is sometimes referred to in the literature in a different sense as a calibration curve. As used herein, “generating a calibration curve” refers to preparing the separate calibrants, as opposed to the mathematical process of generating a calibration curve plot or curve fit based on data obtained by measuring standard samples. A set of calibrants of the invention can also be referred to, e.g., as an array of calibrants.

According to the invention, a set of calibrants is used determine the concentration of an analyte in a sample of interest. The signals produced by a set of standard samples comprising known concentrations of the analyte (calibrants), ranging from below to above the expected concentration, are plotted and subjected to a parametric or non-parametric curve fitting program. Some typical procedures for accomplishing this curve fitting are described in the Examples. The resulting plot may be linear, or it may take another shape. The amount or concentration of the analyte being measured can be determined using the plot, by interpolation.

In embodiments of the invention, the analytes of the set of calibrants comprise one or more unmodified proteins (e.g., c-erbB2, c-erbB3, estrogen receptor, androgen receptor, progesterone receptor, EGFR, VEGFR (KDR, Flk-2), c-met, PDGFR, PDGRα, PDGRβ, FLT3, COX-2, the specific cleavage products listed in the Tables herein, or others); or one or more post-translationally modified proteins (e.g. by phosphorylation, sumolyation, myristylation, farnyslation, acetylation, sufonation, glycosylation, or isoforms that are derived by a specific proteolysis (cleavage) process, such as cleaved caspase 3, or others). In one embodiment, the analytes comprise one or more phosphoprotein isoforms. Phosphoproteins that can be measured (quantitated) by a method of the invention include, e.g., one or more of the phosphoproteins listed in Tables 1, 2, 3, 4 and/or 5 (e.g., c-erbB2(Y1248), EGFR (Y845, Y1045, Y1068, Y1148, Y1173)). A set of calibrants of the invention may comprise, e.g., one or more (e.g., at least about 5, 10, 15, 20, 25, 30 or all 32) of the proteins or protein isoforms listed in Table 1.

TABLE 1

| | | |
|---|---|---|
| 1 | Total EGFR | total epidermal growth factor receptor 1 |
| 2 | p. EGFR (Y1086) | epidermal growth factor receptor 1, with phosphorylation at |
| | | tyrosine residue #1086 |
| 3 | p. EGFR (Y1173) | epidermal growth factor receptor 1, with phosphorylation at |
| | | tyrosine residue #1173 |
| 4 | p. EGFR (Y992) | epidermal growth factor receptor 1, with phosphorylation at |
| | | tyrosine residue #992 |
| 5 | Total erbB2 | total epidermal growth factor receptor 2 |
| 6 | p. erbB2 (Y1248) | epidermal growth factor receptor 2, with phosphorylation at |
| | | tyrosine residue #1248 |
| 7 | Total erbB3 | total epidermal growth factor receptor 3 |
| 8 | p. erbB3 (Y1289) | epidermal growth factor receptor 3, with phosphorylation at |
| | | tyrosine residue #1289 |
| 9 | Total VEGFR | total vascular endothelial growth factor receptor |
| 10 | Total VEGFR2 | total vascular endothelial growth factor receptor 2 |
| | (KDR, Flk-1) | |
| 11 | p. VEGFR2 (Y951) | vascular endothelial growth factor receptor 2, with |
| | | phosphorylation at tyrosine residue #951 |
| 12 | p. VEGFR2 (Y996) | vascular endothelial growth factor receptor 2, with |
| | | phosphorylation at tyrosine residue #996 |
| 13 | p. VEGFR2 (Y1175) | vascular endothelial growth factor receptor 2, with |
| | | phosphorylation at tyrosine residue #1175 |
| 14 | Total PDGFR alpha | total platelet derived growth factor receptor alpha |
| 15 | p. PDGFR alpha | platelet derived growth factor receptor alpha, with |
| | (Y754) | phosphorylation at tyrosine residue #754 |
| 16 | Total PDGFR beta | total platelet derived growth factor receptor beta |
| 17 | p. PDGFR beta | platelet derived growth factor receptor beta, with |
| | (Y751) | phosphorylation at tyrosine residue #751 |
| 18 | Total FLT3 | total FMS-related tyrosine kinase 3 |
| 19 | p. FLT3 | total FMS-related tyrosine kinase 3, with phosphorylation at |
| | (Y589/Y591) | tyrosine residue #589/591 |
| 20 | p. FLT3 (Y842) | activated total FMS-related tyrosine kinase 3, with |
| | | phosphorylation at tyrosine residue #842 |
| 21 | p. Ret (Y905) | activated Ret proto-oncogene receptor tyrosine kinase, with |
| | | phosphorylation at tyrosine residue #905 |
| 22 | p. Src (Y416) | Src tyrosine kinase, with phosphorylation at tyrosine residue |
| | | #416 |
| 23 | p. Akt (S473) | Akt (protein kinase B or Rac), with phosphorylation at serine |
| | | residue #473 |
| 24 | p. Shc (Y317) | Shc, with phosphorylation at tyrosine residue #417 |
| 25 | p. c-Kit (Y719) | c-kit proto oncogene receptor tyrosine kinase, with |
| | | phosphorylation at tyrosine residue #719 |
| 26 | p. c-Abl (Y735) | c-abl proto-oncogene, with phosphorylation at tyrosine residue |
| | | #735 |
| 27 | p. c-Abl (Y245) | c-abl proto-oncogene, with phosphorylation at tyrosine residue |
| | | #245 |
| 28 | p. c-Abl (Y412) | c-abl proto-oncogene, with phosphorylation at tyrosine residue |
| | | #412 |
| 29 | p. Erk 1-2 | p44/42 mitogen activated protein kinase, with phosphorylation at |
| | (T202/Y204) | threonine residue #202/tyrosine residue #204 |
| 30 | p. mTOR (S2448) | mammalian target of rapamycin, with phosphorylation at |
| | | tyrosine residue #2448 |
| 31 | p. mTOR (S2481) | mammalian target of rapamycin, with phosphorylation at |
| | | tyrosine residue #2481 |
| 32 | p. P70 S6 (T389) | p 70 S6 kinase, with phosphorylation at threonine residue #389 |

In a set of calibrants of the invention, the calibrants for each of the analytes may be generated by (i) incubating cells of a suitable cell line with a suitable agent (e.g. a ligand, mitogen or other agent), under conditions such that a high level of the analyte is produced (induced, generated) in the cell, and lysing the cells to generate an upper calibrant;
(ii) incubating the cell line of (i) in the absence of the agent, or incubating, in the presence of the agent, a cell line that is not stimulated by the agent or that is stimulated to a low level, and lysing the cells to generate a lower calibrant; and
(iii) mixing in a series of defined ratios the upper and lower calibrants of (i) and (ii), to generate a series of calibrants containing intermediate amounts of the analyte(s).

The upper, lower and intermediate calibrants are immobilized (e.g., intermingled, distributed, spotted, printed, combined individually) on a surface which contains, or which will contain, samples to be analyzed (e.g. in an RPMA assay), to generate a set of calibrants.

In another embodiment of the invention, the calibrants for each of the analyte(s) are generated by

(i) incubating cells of a first cell line which produce high amounts of the one or more analytes (e.g., the breast cancer line, SKBR3, which overproduces c-erbB2), and lysing the cells to generate an upper calibrant;
(ii) incubating cells of a second cell line which produce low levels or undetectable amounts of one or more the analyte(s) (e.g., the breast cancer cell line, MDA-231, which underexpresses c-erbB2) and lysing the cells to generate a lower calibrant; and
(iii) mixing in a series of defined ratios the upper and lower calibrants of (i) and (ii), to generate a series of calibrants containing intermediate amounts of the analyte(s) (e.g., c-erbB2).

In another embodiment of the invention, the calibrants for the analyte(s) are generated by making a series of dilutions of purified or substantially purified analytes into a cell lysate or bodily fluid which lacks the analyte(s), under conditions such that the total amount of biological components of the lysate or bodily fluid in each dilution is constant in each calibrant.

A set of calibrants of the invention may be used to quantitate at least about 10 (e.g., at least about 20, 40, 60, 80, 100, 200, etc.) analytes, in which case the set of calibrants comprises at least about 10 (e.g., at least about 20, 40, 60, 80, 100, 200, etc.) calibrants, one for each of the analytes. A single calibrant (e.g., a cell lysate) can serve as a calibrant (a combined calibrant) for a plurality (e.g., at least about 20, 40, 60, 80, 100, 200, etc) analytes. The calibrants of a set of calibrants of the invention can comprise, e.g., the markers described in Tables 1-6. For example, incubation of Jurkat cells with FasL or Etoposide, followed by lysis of the cells, gives rise to a (combined) calibrant for at least two phosphoprotein isoforms; incubation of A431 cells with EGF, followed by lysis of the cells, gives rise to a (combined) calibrant for at least 15 phosphoprotein isoforms; incubation of HeLa cells with Pervanadate, followed by lysis of the cells, gives rise to a (combined) calibrant for at least 25 phosphoprotein isoforms; and incubation of Jurkat cells with Calyculin, followed by lysis of the cells, gives rise to a (combined) calibrant for at least 65 phosphoprotein isoforms.

A set of calibrants of the invention may comprise at least about 5 (e.g. about 5-8) spots containing calibrants, each calibrant (spot) containing a different amount of the analyte(s). The amounts of the analytes in the lowest to the highest calibrant in a set of calibrants of the invention can span a range of at least 2 (e.g., at least about 5 or at least about 8) orders of magnitude. In one embodiment of the invention, in which the calibrants comprise phosphoprotein isoforms, at least two of the phosphoprotein analytes may be different phosphoproteins, or at least two of the phosphoprotein analytes may be different isoforms of the same phosphoprotein, which are phosphorylated at different amino acid residues.

Another aspect of the invention is a method for detecting the amount of an (one or more) analyte in a sample from a subject, comprising (a) immobilizing on a surface an aliquot of the sample and a set of calibrants of the invention; (b) contacting the sample aliquot and the calibrants of the set of calibrants with a primary antibody that is specific for the analyte, under conditions effective for the primary antibody to specifically interact with the analyte; (c) detecting the interaction of analyte in the sample aliquot and in the calibrants of the set of calibrants with the primary antibody, using a secondary antibody that is specific for the primary antibody, thereby generating a detectable signal that is proportional to the amount of the analyte in the sample aliquot and in the calibrants; (d) comparing the detectable signal obtained from the aliquot to the detectable signals of the series of corresponding calibrants in the set of calibrants; and, optionally, (e) interpolating the amount of signal from the analyte in the sample to a non-parametrically or parametrically determined curve fit of the detectable signals of the calibrants in the set of calibrants, thereby determining the concentration of the analyte in the sample

Another aspect of the invention is a method for detecting the amount of each of at least 3 analytes in a sample (e.g., lysed tissue or cells) from a subject, comprising

a) immobilizing on each of at least 3 separate (independent) surfaces an aliquot of the sample (e.g. at a defined position),
wherein each of the at least 3 surfaces is designated for detecting the amount of one of the at least 3 analytes, and
immobilizing on each of the at least 3 surfaces a set of calibrants of the invention;

b) contacting the sample aliquots and the calibrants of the set of calibrants on each of the at least 3 surfaces with a primary antibody that is specific for the analyte to be detected on that surface, under conditions effective for the primary antibody to specifically interact with (bind to) the analyte;

c) detecting the interaction of analytes in the sample aliquot and in calibrants of the set of calibrants with the primary antibodies, using a secondary antibody that is specific for the primary antibodies and which is labeled with a detectable moiety that has a dynamic range of at least two orders of magnitude, thereby generating detectable signals that are proportional to the amounts of the analytes in the sample aliquots and in the calibrants; and

d) comparing the detectable signal obtained from each aliquot to the detectable signals of the series of corresponding calibrants in the set of calibrants.

As used herein, the singular forms “a,” “an” and “the” include plural referents unless the context clearly dictates otherwise. For example, “an” aliquot of the sample, as used above, includes 2, 3, 4, 5 or more aliquots of the sample (e.g., duplicates or dilutions of one aliquot).

The steps of a method of the invention are not limited to being conducted in any particular order. For example, in a), the set of calibrants can be immobilized on the surface before, at essentially the same time as, or after the sample aliquots are immobilized on the surface.

This method can further comprise

e) interpolating the amount of signal from each analyte in the sample to non-parametrically or parametrically determined curve fit of the detectable signals of the calibrants in the set of calibrants, thereby determining the concentration of the analytes in the sample.

Another aspect of the invention is a method for detecting the amount of just one analyte in a sample from a subject. In this embodiment, an aliquot of the sample is immobilized on one surface, and the method is carried out as above. To analyze two analytes, aliquots of the sample are immobilized on two surfaces, and so on.

In a method of the invention, a single (neat) aliquot of a sample can be immobilized on each slide, or two aliquots of the sample can be immobilized: a first aliquot which is undiluted (neat) and an additional aliquot, which is diluted. Each aliquot can be present in a sufficient number of replicates (e.g., duplicates or triplicates) for statistical robustness. In embodiments of the invention, when a sample is a lysed tissue sample, the second aliquot can be immobilized which is diluted, e.g., to as much as, but no more than, about 1:10. A sample that is a bodily fluid can be diluted, e.g., to as much as, but no more than, about 1:20.

In embodiments of this method, the analytes may be phosphoprotein isoforms which form part of one or more kinase signaling pathways.

In a method of the invention, the calibrants of the set of calibrants may be immobilized on the surface before, at substantially the same time as, or after the sample aliquots are immobilized on the surface.

At least one analyte (e.g., at least about 5, 10, 20, 40, 60, 80, 100, or 200; or between about 1-20 or 7-20) may be detected for each sample. The number of samples that are analyzed (immobilized on a surface) to determine the amounts of the analytes present therein can range from 1 to at least about 200 (e.g., at least about 500 or 1,000). Current technology allows one to immobilize a total of about 1,000-3,000 spots on a slide. Thus, if each aliquot is spotted one time as a neat spot and in one dilution, one can assay aliquots from about 500-1500 samples on a single slide; if each sample is spotted 4 times (e.g., neat and in one dilution, each in duplicate), one can assay about 250-750 samples on a single slide, and so on. The number of samples that can be analyzed may increase as technology improves.

A sample which is assayed by a method of the invention can be from a variety of sources, including a tissue or a bodily fluid of a subject. In one embodiment, at least one (e.g. all) of the samples is a tissue sample that has been procured by laser microdissection under microscopic visualization, followed by lysis and fixation, and a volume of less than 20 μL, comprising the cellular contents of equal to or less than 1500 cells, is immobilized on the surface. In this embodiment, if the tissue sample comprises phosphoproteins, it can be prepared in a composition comprising (a) a preservative/fixative that is effective to fix the phosphoproteins in the sample, and that has a sufficient water content to be soluble for a stabilizer and/or a permeability enhancing agent; (b) a stabilizer, comprising (i) a kinase inhibitor and (ii) a phosphatase inhibitor, and, optionally, (iii) a protease inhibitor; and (c) a permeability enhancing agent.

In a method of the invention, the detectable moiety labeling the secondary antibody may be, e.g., a fluorescent, chemoluminescent, or chemifluorescent dye, e.g. a near-infra-red dye (such as an IRDye® Infrared Dye Optical Agent from LI-COR Biosciences). Generally, the detectable moiety labeling the secondary antibody is selected such that the background autofluorescence of the surface is negligible and the detectable moiety is not easily quenched over time.

The surface to which aliquots are immobilized can comprise, e.g., nitrocellulose, such as a glass-backed nitrocellulose slide. In one embodiment, at least about 16 (e.g., between about 16 and 100) surfaces are present as separate nitrocellulose sectors on a larger surface (such as a glass slide).

Another embodiment of the invention is in a method of RPMA of at least 1 (e.g., at least 3) analytes, the improvement comprising

a. immobilizing on each surface comprising samples to be analyzed a set of calibrants of the invention, and

b. labeling the secondary antibody with a dye that has a dynamic range of at least two orders of magnitude,

wherein the calibrants in the set of calibrants span a concentration range of at least two orders of magnitude.

Another aspect of the invention is a kit for performing a method of the invention (e.g., a calibrated RPMA assay), comprising (a) a set of calibrants of the invention, wherein the calibrants are stored as frozen liquids at about 80° C. or as lyophilized samples; or (b) a surface (e.g. a slide) on which are immobilized a set of calibrants of the invention, which is stored at about 80° C.

An embodiment of the invention includes an “array,” e.g. part of a reverse phase microarray, for determining the amount of an analyte in a sample, comprising a set of calibrants immobilized on a surface, the calibrants defining the range of a set of calibrants for determining the amount of the analyte, the set comprising an upper and a lower calibrant, wherein each of the calibrants comprises a predetermined amount of the analyte and a biological fluid at the same dilution. The calibrants include differing amounts of analyte in a diluent having a constant concentration of biological components. The calibrants are not diluted with different amounts of buffer.

Another embodiment is a set of calibrants useful for preparing an array (of calibrants) as above.

Another embodiment is a method of preparing the calibrants, comprising producing an upper calibrant and a lower calibrant, and optionally combining the upper and lower calibrants to produce intermediate calibrants.

Another embodiment of the invention is a method for preparing an array of calibrants, as above, comprising immobilizing the calibrants on a surface.

Another embodiment of the invention is a method of using an array of calibrants as above, comprising immobilizing the array of calibrants and one or more aliquots of samples on a surface (e.g. in the form of an array), and contacting the calibrants and aliquots of samples with reagents to generate a signal, measuring the signal, and using the signal generated by the calibrants to determining the amount of analyte present.

A method of the invention can be used to quantitate any analyte for which there is an antibody that binds specifically, and which can thus be subjected to immunological analysis, such as RPMA analysis. By an antibody that “specifically” interacts with (or binds to) an analyte is meant an antibody that has a higher affinity, e.g., a higher degree of selectivity, for the analyte than for other analytes in a sample. The degree of selectivity should be sufficiently large to allow an investigator to determine the presence and amount of an analyte of interest in a sample (such as a tissue lysate or a bodily fluid) that also comprises potentially competing targets. The affinity or degree of specificity of an antibody can be determined by a variety of routine procedures, including, e.g., competitive binding studies. For example, an antibody of the invention may bind at least about 25% to 100 fold, or more, as efficiently to an analyte of interest than it binds to other analytes in a sample, such as a sample from a cell lysate.

Among the types of molecules that can be analyzed/quantitated by a method of the invention are a variety of non-proteinaceous molecules, such as nucleic acids (DNA, RNA, etc), lectins, drugs/chemical entities, metabolites, etc. Other suitable non-proteinaceous analytes will be evident to the skilled worker.

Examples of suitable analytes include unmodified proteins, such as, e.g., c-erbB2, c-erbB3, estrogen receptor, androgen receptor, progesterone receptor, EGFR, VEGFR (KDR, Flk-2), c-met, PDGFR, PDGRα, PDGRβ, FLT3, COX-2 or others that will be evident to a skilled worker; or proteins which exhibit post-translational modifications such as, e.g., phosphorylation, sumolyation, myristylation, farnyslation, acetylation, sufonation, glycosylation, or specific proteolysis (cleavage), such as cleavage at specific sites of caspase 3. Much of the discussion herein is directed to proteins which are phosphorylated at particular residues (phosphoprotein isoforms), but it is to be understood that unmodified proteins, or proteins having other types of post-translational modification, are also included.

Antibodies are readily available that are specific for a variety of unmodified proteins, or for proteins that are modified at specific residues with any of the post-translational modifications discussed above, or others. For example, a large number of antibodies are commercially available that specifically recognize individual phosphoprotein isoforms having a particular phosphorylated amino acid residue, or peptides which contain that modification. Such antibodies are available, e.g. from Cell Signaling Technology, Danvers, Mass.; Upstate-Millipore, N.J.; LabVision, Freemont, Calif.; Invitrogen-Biosource, Carlsbad, Calif.; BD, Franklin Lakes, N.J.; or other sources. See, e.g., Wulfkuhle et al. “Multiplexed Cell Signaling Analysis of Human Breast Cancer: Applications for Personalized Therapy,” J of Prot Res. Feb. 8, 2008 epub ahead of print, for a description of phosphoproteins for which there are commercially available antibodies. The same, and other, companies provide antibodies against isoforms of other types of post-translationally modified proteins.

Alternatively, a desired antibody can be generated using routine, conventional procedures. For example, a synthetic peptide comprising a phosphorylation site from a phosphoprotein isoform of interest (either in the non-phosphorylated or phosphorylated form) can be used as an antigen to prepare a mixture of antibodies. Antibodies are then selected and verified which detect only the version of the protein which is phosphorylated at the residue of interest, but not the non-phosphorylated version of the protein, and vice-versa. It will be readily apparent to one skilled in the art that antibodies generated against (specific for) any protein or post-translationally modified protein can be prepared using well-established methodologies (e.g., the methodologies described by Harlow et al. in Antibodies. A Laboratory Manual, Cold Spring Harbor Laboratory, Cold Spring Harbor, 1988, pp. 1-725).

For general references describing methods of molecular biology which are mentioned in this application, see, e.g., Sambrook, et al. (1989), Molecular Cloning, a Laboratory Manual, Cold Harbor Laboratory Press, Cold Spring Harbor, N.Y.; Ausubel et al. (1995). Current Protocols in Molecular Biology, N.Y., John Wiley & Sons; Davis et al. (1986), Basic Methods in Molecular Biology, Elsevier Sciences Publishing Inc., New York; Hames et al. (1985), Nucleic Acid Hybridization, IL Press; Dracopoli et al. Current Protocols in Human Genetics, John Wiley & Sons, Inc.; and Coligan et al. Current Protocols in Protein Science, John Wiley & Sons, Inc.

An antibody for use in a method of the invention may be, e.g., polyclonal; monoclonal; a single chain monoclonal antibody; a whole (full size, bivalent) antibody, such as an IgG antibody; a suitable antibody fragment; a miniantibody; an antibody fusion product; humanized; and/or human.

“Subjects” (e.g., patients) from which samples can be analyzed include, e.g., laboratory animals (such as mouse, rat, rabbit, or guinea pig), farm animals, and domestic animals or pets (such as a cat or dog). Non-human primates and human patients are included.

“Samples” to be analyzed by a method of the invention can be prepared from tissues, cells or bodily fluids from a subject. Suitable samples include, e.g., biopsy samples (such as a tumor biopsies, fine needle aspiration, etc.), primary tissue, metastatic tissue, bodily fluids (such as blood, serum, tears, vitreous humor, urine, sweat, saliva, peritoneal washings, bronchial lavage, nipple fluid aspirate, semen, or the like), products of a cell (e.g., proteins that are secreted, excreted, shed, etc. from a cell, which can be present, e.g., in conditioned medium, cellular exudate recovered from cell washings, etc.), low-molecular weight fraction, etc. Other suitable sources for samples include, e.g., lysates derived from peripheral blood lymphocytes, magnetic sorted cells, FACS sorted cells, cell lines in culture, xenograft tissues, etc.

Phrases such as “a sample containing an analyte” or “detecting or quantitating an analyte in a sample” are not meant to exclude samples or determinations (detection attempts) wherein no analyte is contained or detected. In a general sense, this invention involves a method to determine whether an analyte is present in a sample, irrespective or whether it is detected or not.

Samples can be lysed or otherwise prepared for immunological analysis by conventional methods. For example, a tissue sample may be procured by laser microdissection under microscopic visualization, lysed, and fixed; and a volume of less than about 2-100 (e.g. less than about 20) μL, comprising the cellular contents of equal to or less than about 100-100,000 (e.g., less than about 1500) cells, may be immobilized on a surface. In one embodiment of the invention, in which the analytes comprise phosphoproteins, a tissue sample is fixed in a composition comprising (a) a fixative/preservative that is effective to fix the phosphoproteins in the sample, and that has a sufficient water content to be soluble for a stabilizer and/or a permeability enhancing agent; (b) a stabilizer, comprising (i) a kinase inhibitor and (ii) a phosphatase inhibitor, and, optionally, (iii) a protease inhibitor; and (c) a permeability enhancing agent. Examples of such compositions are described in co-pending application PCT/US07/22744, incorporated herein by reference.

Aliquots of samples to be analyzed (e.g., cell lysates or body fluids) are immobilized on a suitable surface. In one embodiment of the invention, aliquots are immobilized at defined, spatially discrete, and addressable or identifiable positions on a suitable surface. This defined immobilization can facilitate automated (e.g., mechanized, robotic) analysis of the aliquots. The terms bound, attached, contacted with, spotted, or printed are sometimes used herein to describe ways of immobilizing a sample on a surface. Samples can be attached to a surface covalently or non-covalently.

Aliquots of a cell lysate or body fluid input sample are printed in neat undiluted spots in a sufficient number of replicates (e.g. duplicates, triplicates, etc.) for statistical robustness. The number of aliquots to be printed can be determined empirically, using routine, conventional procedures, as well as statistically, based on the variance of the assay. Optionally, one or more dilutions of the sample are also printed, in a sufficient number of replicates for statistical robustness. For example, the dilution can be at least about 1:4, at least about 1:10, etc. For the detection of analytes in samples such as blood, in which there are high concentrations of potential contaminants, the sample can be diluted at least about 1:200 prior to printing an aliquot. For a blood sample, for example, this high dilution can reduce non-specific binding of the primary and secondary antibodies to ultra-high concentration resident blood analytes, such as immunoglobulin and albumin. Using a diluted second spot provides a back-up in case of fluorescence saturation in the neat spot. This provides an opportunity to analyze a sample if abnormally and unpredictably high quantities of a given analyte are present, without sacrificing large quantities of the input sample.

Any suitable surface can be used in a method of the invention. The surface (usually a solid) can be any of a variety of organic or inorganic materials or combinations thereof, including, merely by way of example, plastics such as polypropylene or polystyrene; ceramic; silicon; (fused) silica, quartz or glass, which can have the thickness of, for example, a glass microscope slide or a glass cover slip; paper, such as filter paper; diazotized cellulose; nitrocellulose filters; nylon membrane; or polyacrylamide gel pad. Substrates that are transparent to light are useful when the method of performing an assay involves optical detection. In embodiments of the invention, the surface is nitrocellulose or a glass-backed nitrocellulose slide. In one embodiment, there are at least about 16 (e.g., between about 16 and 100) separate surfaces, which are nitrocellulose sectors present on a larger surface, such as a glass slide; each of the separate surfaces can be used to analyze (quantitate) a different analyte.

The shape of the surface is not critical. It can, for example, be a flat surface such as a square, rectangle, or circle; a curved surface; or a three dimensional surface such as a bead, particle, strand, precipitate, tube, sphere; etc.

Following the immobilization of aliquots of samples (and of calibrants) onto one or more surfaces, each surface is contacted with an antibody that is specific for the analyte to be assayed on that surface. Conditions that are effective for specific binding of an antibody to an analyte of interest in the sample can be determined empirically, using routine, conventional procedures.

Following the interaction (e.g., binding) of a primary antibody as described above to an analyte of interest in one or more samples on a surface, a secondary antibody is contacted with the surface, under conditions that are effective for a specific interaction (e.g. binding) to the primary antibody. For example, if the first antibody is produced in rabbits, a goat anti-rabbit antiserum can be used for the secondary antibody.

In a method of the invention, the secondary antibody comprises a detectable moiety which has a dynamic range of at least two orders of magnitude. A skilled worker will recognize a variety of suitable detectable moieties, e.g., fluorescent, chemoluminescent, or chemifluorescent dyes. In one embodiment, a fluorescent dye coupled secondary antibody is used as the detection generating system. The fluorophore is chosen such that background autofluorescence of the substratum is negligible and the fluorophore does not easily quench with exposure. The fluorophore is also chosen such that autofluorescence of the input sample (e.g. serum fluorescence due to hemoglobin, etc) is minimized. Suitable fluorophores include the near-infra red dyes from LICOR Biosciences, which do not excite/emit within wavelengths that nitrocellulose does, and do not easily quench over time.

The dynamic range of the signal generated by the detectably labeled moiety, such as a fluorophore, may be as large as about 5-8 orders of magnitude, allowing for the detection and quantitation of analytes in a sample spanning a wide range of concentrations. The intensity of the signal can also be varied by exposing the surface to shorter or longer times, in order to increase or decrease sensitivity.

A variety of methods can be used to generate set of calibrants containing calibrants for one or more analytes of interest, in a series of defined concentrations. For example, “upper” calibrants can be generated by contacting (stimulating, inducing) any of a variety of cells lines with any of a variety of agents that stimulate (induce) production of an analyte to be quantitated, and then lysing the cells. A “lower” calibrant can be produced by inducing the cell line with an agent that is less effective, generating a lower level of the analyte, or by inducing a different cell line with the agent, wherein the different cell line produces low or undetectable amounts of the analyte, and then lysing the cells. Alternatively, an unstimulated cell line can be used, which produces low or undetectable amounts of the analyte.

Mixtures of the lower calibrant and the upper calibrant are then prepared, in various defined ratios, to generate a series of calibrants having a range of intermediate values of the analyte. For example, mixtures can be generated with ratios of the upper and lower calibrants of 0:100, 5:95, 10:90, etc. through 90:10, 95:10 and 100:0. In this series of ratios, the percentages of the analyte are 0%, 5%, 10% . . . through 90%, 95% and 100%, yet the total amount of biological components (from the cell lysates) is constant in all of the calibrants. Any desired series of ratios (e.g., serial dilutions) can be used. In one embodiment, the upper calibrant is diluted about 10-fold, 25-fold, 50-fold, or more.

One analyte may be present in a high amount in a particular calibrant, but another analyte may be present in a low amount in that calibrant. Thus, an “upper” calibrant for one of the analytes may serve as a “lower” calibrant for the other analyte, and vice-versa.

The calibrants having different amounts of an analyte of interest are immobilized on (e.g., spotted onto, printed onto) each surface comprising samples to be analyzed, in any pattern that is desired. A set of calibrants may comprise any desired number of the calibrants having different amounts of an analyte of interest. For example, the curve may comprise at least about 5 of these calibrants, e.g. between about 5-15, between about 8-15, etc. The term “about,” as used herein, means plus or minus 20%. Thus, about 5 calibrants means 4-6 calibrants. When the value is not an integer, it will be evident to a skilled worker that the nearest integer is meant. For example, about 6 calibrants is not 4.8 to 7.2, but is 5-7 calibrants. The endpoints of ranges, as used herein, are included within that range. For example, a range of 5-7 calibrants (or between 5-7 calibrants) includes both 5 and 7 calibrants. The range of amounts of the analyte of interest in a set of calibrants, from the lowest to the highest amount, may span at least about two orders of magnitude, e.g., at least about five, eight, ten or more orders of magnitude.

In one embodiment of the invention, a set of calibrants is generated for quantitating the amount of one or more phosphoprotein isoforms in a sample. For example, cell lines can be incubated with any of a variety of mitogens, which stimulate the activation (phosphorylation) of cellular proteins. The proteins which are activated can be members of one or more signaling pathways or cascades. The tables below summarize some of the proteins which are phosphorylated, and the amino acid residue at which they are phosphorylated, following stimulation of a particular cell line with a particular mitogen. An investigator wishing to quantitate one of the phosphoprotein isoforms listed in one of the tables below can select an appropriate cell/stimulant combination to use for generating a suitable calibrant for that analyte. Other combinations of cells/stimulating agents will be evident to a skilled worker. If an investigator wishes to quantitate the amount of analytes that are found in more than one of these tables, two or more sets of calibrants can be used to generate a combined set of calibrants. In one embodiment, two or more calibrants are mixed together, and dilutions are then made to generate a mixed set of calibrants. Generally, however, it is preferable to immobilize independent sets of calibrants separately. See, e.g., Example II, in which 3 independent sets of calibrants are used, to quantitate 26 endpoints.

TABLE 2

| Endpoint | Suitable Calibrant Lysate |
|---|---|
| 4E-BP1 (T37/46) | Jurkat + Calyculin |
| 4E-BP1 (T70) | Jurkat + Calyculin |
| c-Abl (T735) | Jurkat + Calyculin |
| Acetyl-CoA Carboxylase (S79) | Jurkat + Calyculin |
| Adducin (S662) | Jurkat + Calyculin |
| Akt (S473) | Jurkat + Calyculin |
| Akt (T308) | Jurkat + Calyculin |
| AMPKalpha1 (S485) | Jurkat + Calyculin |
| AMPKBeta1 (S108) | Jurkat + Calyculin |
| ASK1 (S83) | Jurkat + Calyculin |
| ATF-2 (T71) | Jurkat + Calyculin |
| ATF-2 (T69/71) | Jurkat + Calyculin |
| ATP-Citrate Lyase (S454) | Jurkat + Calyculin |
| Bad (S112) | Jurkat + Calyculin |
| Bad (S136) | Jurkat + Calyculin |
| Bcl-2 (S70) (5H2) | Jurkat + Calyculin |
| Bcl-2 (T56) | Jurkat + Calyculin |
| Catenin (beta) (S33/37/T41) | Jurkat + Calyculin |
| Chk1 (S345) | Jurkat + Calyculin |
| Chk2 (S33/35) | Jurkat + Calyculin |
| Cofilin (S3) (77G2) | Jurkat + Calyculin |
| CREB (S133) | Jurkat + Calyculin |
| eNOS (S113) | Jurkat + Calyculin |
| eNOS (S1177) | Jurkat + Calyculin |
| eNOS/NOS III (S116) | Jurkat + Calyculin |
| ERK 1/2 (T202/Y204) | Jurkat + Calyculin |
| Estrogen Receptor alpha (S118) (16J4) | Jurkat + Calyculin |
| FADD (S194) | Jurkat + Calyculin |
| FKHR (S256) | Jurkat + Calyculin |
| FKHRL1 (S253) | Jurkat + Calyculin |
| FKHR (T24)/FKHRL1 (T32) | Jurkat + Calyculin |
| GSK-3alpha/beta (S21/9) | Jurkat + Calyculin |
| IkappaB-alpha (S32/36) (5A5) | Jurkat + Calyculin |
| IRS-1 (S612) | Jurkat + Calyculin |
| Lck (Y505) | Jurkat + Calyculin |
| LKB1 (S428) | Jurkat + Calyculin |
| MARCKS (S152/156) | Jurkat + Calyculin |
| MEK1/2 (S217/221) | Jurkat + Calyculin |
| mTOR (S2448) | Jurkat + Calyculin |
| mTOR (S2481) | Jurkat + Calyculin |
| NF-kappaB p65 (S536) | Jurkat + Calyculin |
| p27 (T187) | Jurkat + Calyculin |
| p38 MAP Kinase (T180/Y182) | Jurkat + Calyculin |
| p40 phox (T154) | Jurkat + Calyculin |
| p70 S6 Kinase (T389) | Jurkat + Calyculin |
| PDK1 (S241) | Jurkat + Calyculin |
| PKA C (T197) | Jurkat + Calyculin |
| PKC alpha (S657) | Jurkat + Calyculin |
| PKC alpha/beta II (T638/641) | Jurkat + Calyculin |
| PKC (pan) (betaII S660) | Jurkat + Calyculin |
| PKC delta (T505) | Jurkat + Calyculin |
| PKC theta (T538) | Jurkat + Calyculin |
| PKC zeta/lambda (T410/403) | Jurkat + Calyculin |
| cPLA2 (S505) | Jurkat + Calyculin |
| PLCgamma1 (Y783) | Jurkat + Calyculin |
| PLK1 (T210) | Jurkat + Calyculin |
| PRAS40 (T246) | Jurkat + Calyculin |
| B-Raf (S445) | Jurkat + Calyculin |
| Smad2 (S465/467) | Jurkat + Calyculin |
| Stat3 (S727) | Jurkat + Calyculin |
| RSK3 (T356/S360) | Jurkat + Calyculin |
| S6 Ribosomal Protein (S235/236) (2F9) | Jurkat + Calyculin |
| S6 Ribosomal Protein (S240/244) | Jurkat + Calyculin |
| SAPK/JNK (T183/Y185) | Jurkat + Calyculin |
| SEK1/MKK4 (S80) | Jurkat + Calyculin |

TABLE 3

| | |
|---|---|
| Pyk2 (Y402) | HeLa + PerVan |
| Shc (Y317) | HeLa + PerVan |
| Src Family (Y416) | HeLa + PerVan |
| Stat1 (Y701) | HeLa + PerVan |
| Jak2 (Y1007/1008) | HeLa + PerVan |
| c-Kit (Y703) | HeLa + PerVan |
| c-Kit (Y719) | HeLa + PerVan |
| c-Kit (Y721) | HeLa + PerVan |
| Met (Y1234/1235) | HeLa + PerVan |
| IGF-1 Rec (Y1131)/Insulin Rec (Y1146) | HeLa + PerVan |
| IGF-1R (Y1135/36)/IR (Y1150/51) | HeLa + PerVan |
| (19H7) | |
| Etk (Y40) | HeLa + PerVan |
| eIF4G (S1108) | HeLa + PerVan |
| c-Abl (Y245) | HeLa + PerVan |
| Caspase-9, cleaved (D315) | HeLa + PerVan |
| Caspase-9, cleaved (D330) | HeLa + PerVan |
| EGFR (Y845) | HeLa + PerVan |
| EGFR (Y1045) | HeLa + PerVan |
| Stat5 (Y694) | HeLa + PerVan |
| Stat6 (Y641) | HeLa + PerVan |
| VEGFR 2 (Y951) | HeLa + PerVan |
| VEGFR 2 (Y996) | HeLa + PerVan |
| ErbB3/HER3 (Y1289) (21D3) | HeLa + PerVan |
| Paxillin (Y118) | HeLa + PerVan |
| PDGF Receptor beta (Y751) | HeLa + PerVan |

TABLE 4

| | |
|---|---|
| Src (Y527) | A431 + EGF |
| c-Raf (S338) (56A6) | A431 + EGF |
| Ras-GRF1 (S916) | A431 + EGF |
| EGFR (Y992) | A431 + EGF |
| MAPK (pTEpY) | A431 + EGF |
| EGFR (Y1068) | A431 + EGF |
| EGFR (Y1148) | A431 + EGF |
| EGFR (Y1173) | A431 + EGF |
| Akt1/PKB alpha (S473) (SK703) | A431 + EGF |
| Bad (S155) | A431 + EGF |
| FAK (Y397) (18) | A431 + EGF |
| FAK (Y576/577) | A431 + EGF |
| c-erbB2/HER2 (Y1248) | A431 + EGF |
| PDGF Receptor beta (Y716) | A431 + EGF |
| p70 S6 Kinase (T412) | A431 + EGF |
| PAK1 (S199/204)/PAK2 (S192/197) | A431 + EGF |
| A-Raf (S299) | A431 + EGF |

TABLE 5

| | |
|---|---|
| MSK1 (S360) | HeLa Untreated |
| p70 S6 Kinase (S371) | HeLa Untreated |
| VEGFR 2 (Y1175) (19A10) | HeLa Untreated |
| 4E-BP1 (S65) | HeLa Untreated |
| Catenin (beta) (T41/S45) | HeLa Untreated |
| Elk-1 (S383) | HeLa Untreated |
| PKR (T446) | HeLa Untreated |
| PTEN (S380) | HeLa Untreated |

TABLE 6

| | |
|---|---|
| PARP, cleaved (D214) | Jurkat + FasL |
| Caspase-3, cleaved (D175) | Jurkat + FasL |
| Caspase-6, cleaved (D162) | Jurkat + Etoposide |
| Caspase-7, cleaved (D198) | Jurkat + Etoposide |

In one embodiment of the invention, a set of calibrants is generated for quantitating phosphoproteins which are phosphorylated on threonine and/or serine residues, by treating a suitable cell line with a serine/threonine phosphatase inhibitor, such as calyculin; or for quantitating phosphoproteins which are phosphorylated on tyrosine residues, by treating a suitable cell line with a tyrosine phosphatase inhibitor, such as pervanadate.

In other embodiments of the invention, sets of calibrants are generated to quantitate proteins which comprise other posttranslational modifications, such as sumolyation, myristylation, farnyslation, acetylation, sufonation, or glycosylation. A variety of combinations of cells and stimulatory agents (such as ligands) can be used, such as those provided above.

In other embodiments, sets of calibrants are generated by preparing lysates of cells from cell lines which produce different levels of the analyte(s) of interest. An upper calibrant and a lower calibrant generated in this manner can be mixed in different ratios to generate calibrants having intermediate amounts of the analytes(s). This method can be used, e.g, to generate sets of calibrants for quantitating proteins which do not comprise post-translational modifications. For example, to quantitate the amount of c-erbB2 in a sample, one can use as an upper calibrant a lysate of a cell line which produces large amounts of the protein. One suitable cell line is SKBR 3, which is known to have approximately 2,390,000±130,000 c-erbB2 receptors/cell, corresponding to a 3+ immunohistochemical score. As a lower calibrant, one can use a lysate of a cell line which produces low or undetectable amounts of the protein. A suitable cell line is MDA231, which is known to have approximately 21,600±6700 c-erbB receptors/cell, corresponding to a 0 immunohistochemical score. The generation of a set of calibrants for the quantitation of c-erbB is discussed in Example III. The accurate quantitation of the amount of c-erbB2 (HER2) in a sample from a subject having metastatic breast cancer can be useful for determining whether the subject will be responsive to drugs, such as Herceptin, trastuaumab, lapatinib, which target that protein.

Another way to generate a set of calibrants which comprises reference amounts of analytes of interest is to dilute known quantities of purified, substantially purified, or partially purified analytes to cover a desired range of concentrations, in such a way that the total amount of protein in each dilution is kept constant. For example, the analytes can be diluted into cell lysates which do not contain measurable amounts of the analyte(s) to be quantitated. The term “substantially purified”, as used herein refers to a molecule, such as a protein, that is substantially free of other proteins, lipids, carbohydrates, nucleic acids and other biological materials with which it is naturally associated. For example, a substantially pure molecule, such as a protein, can be at least about 60%, by dry weight, preferably at least about 70%, 80%, 90%, 95%, or 99% the molecule of interest. In general, an analyte, such as a protein, that is used in a set of calibrants of the invention, should be sufficiently pure that its concentration is well defined and reproducible.

Calibrants of the invention can be generated at the same time as the samples to be assayed, or they can be prepared in advance and stored. For example, cell lysates—upper, lower and/or one or more of a set of intermediate calibrants—can be stored lyophilized, under an inert gas at room temperature, or as frozen liquids (e.g. at about −80° C.). Alternatively, calibrants can be immobilized on a surface, such as a slide, and stored frozen (e.g. at about −80° C.).

Calibrants can be immobilized on a surface in any of a variety of orientations. In one embodiment, calibrants representing a range of different amounts of analyte(s) of interest are arranged linearly, to facilitate the comparison of the signal from an aliquot of a sample to the range of signals in the set of calibrants which correspond to different amounts/concentrations of the analytes. In other embodiments, particularly when the calibrants are detected robotically with the use of a computer, the calibrants can be arranged in any pattern, provided that they are present at defined positions which can be recognized by the robot/computer.

Independent positive and negative quantitation standards can also be immobilized on a surface. For example, high or low control samples can be used, which are known to comprise high or low amounts of an analyte of interest. These can be from patient samples or well-characterized cell lines, or they can be purified or substantially purified proteins.

Assays to measure analytes of interest by a method of the invention can readily be adapted to high throughput formats. For example, a large number of samples (e.g., as many as 1,000, 3,000, or more) can be assayed rapidly and concurrently for one analyte of interest on a first surface. Furthermore, samples on many additional surfaces can be assayed simultaneously, allowing for the simultaneous assay of many additional analytes. The advent of ever more sophisticated tools, e.g. for spotting/printing samples onto a surface, robotics, improved dispensers and sophisticated detection systems and data-management software has and will allow the analysis of increasing numbers of samples and analytes by a method of the invention.

Calibrated RPMA assays of the invention can be used in a variety of ways, which will be evident to a skilled worker. For example, samples from a subject can be assayed to determine the amount of activation (phosphorylation) of one or more (e.g., 100 or more) proteins whose activation has been shown to be correlated with a pathological condition or disease of interest, or the response to an agent of interest. For example, the activation of members of kinase signaling pathways may be assayed in this manner. This analysis can be used both as a way for diagnosing the condition and as a way of identifying potential therapeutic targets. Methods of the invention can also be used for following the course of a disease or pathologic condition, in drug screening procedures, or the like.

In one embodiment of the invention, one or more of the following non-translationally modified proteins (analytes) are assayed by a method of the invention: c-erbB2, c-erbB3, estrogen receptor, androgen receptor, progesterone receptor, EGFR, VEGFR (KDR, Flk-2), c-met, PDGFR, PDGRα, PDGRβ, FLT3, COX-2, the specific cleavage products listed in the Tables herein, or others. c-ErbB2 stands for erythroblastic leukemia viral oncogene homolog 2, and is sometimes referred to as HER2 (Human Epidermal growth factor Receptor 2). In another embodiment, one or more of the proteins assayed contains one of the following post-translational modifications: phosphorylation, sumolyation, myristylation, farnyslation, acetylation, sufonation, glycosylation, or isoforms that are derived by a specific proteolysis (cleavage) process, such as cleaved caspase 3, or others). In one embodiment, the analytes comprise one or more phosphoprotein isoforms, e.g., one or more of the phosphoproteins listed in Tables 1, 2, 3, 4 and/or 5, including c-erbB2(Y1248) and/or EGFR (Y845, Y1045, Y1068, Y1148 and/or Y1173). In another embodiment, one or more (e.g., at least about 5, 10, 15, 20, 25, 30 or all 32) of the analytes listed in Table 1 are assayed by a method of the invention.

The analyte concentration may be reported and displayed in any suitable fashion. For example, displays of patient values and reference values may be as described in PCT/US2008/003968, filed Mar. 27, 2008, which is incorporated by reference herein. For example, the analyte concentrations can be displayed to highlight those values which fall outside of a reference standard range of values (e.g., in tabular form or in a diagrammatic form). If desired, the results of the assay can be reported to an interested party, such as a physician.

In another embodiment of the invention, a single protein of interest, such as c-erbB2 (HER2), is measured. The accurate quantitation of this protein in a sample from a patient having malignant breast cancer allows a clinician to determine if the patient is likely to be responsive to drugs such as Herceptin, which target this receptor.

Another aspect of the invention is a kit useful for carrying out a method disclosed herein. Such a kit can comprise, e.g., individual calibrants; components for generating calibrants (such as a cell line that produces a high or low amount of an analyte of interest; a cell line and/or a suitable ligand or other agent that will stimulate production of an analyte of interest in the cell line; or the like); or surfaces such as slides which comprise one or more pre-formed sets of calibrants. A skilled worker will recognize other components that can be present in a kit of the invention.

Optionally, a kit of the invention may comprise instructions for performing the method. Other optional elements of a kit of the invention include suitable buffers or other reagents for carrying out the steps of a method of the invention; primary and/or secondary antibodies; containers, or packaging materials. The reagents of the kit may be in containers in which the reagents are stable, e.g., in lyophilized form or stabilized liquids. The reagents may also be in single use form, e.g., in a form for carrying out a single assay.

In the foregoing and in the following examples, all temperatures are set forth in uncorrected degrees Celsius; and, unless otherwise indicated, all parts and percentages are by weight.

### EXAMPLES

### Example I

### Materials and Methods

Methods for preparing, assaying, and analyzing samples for a method of the invention are conventional and well-known to skilled workers. Some of these methods are described below:

Laser Capture Microdissection (of tumor cells): 8 um frozen sections are prepared on either glass or membrane slides. Frozen sections are fixed in 70% ethanol, stained with Mayer's Hematoxylin and Scott's Tap Water Substitute, and dehydrated in gradient ethanol, with a final clearing in xylene.

The slides are rapidly air dried and tumor cells are isolated by laser capture microdissection (Pixcell™ and Veritas™, Arcturus Molecular Devices; CA, USA). Formalin or alcohol fixed paraffin embedded specimens can also be used, and subjected to Laser Capture Microdissection as above with appropriate tissue stains.

Reverse Phase Protein Microarrays. Microdissected cells are subjected to lysis in boiling 2.5% beta-mercaptoethanol in T-PER (Pierce, Rockford, Ill.) mixed 1:1 with 2×SDS Tris-glycine buffer (Invitrogen, Carlsbad, Calif.). Reverse phase protein microarrays are printed with whole cell protein lysates as described by Sheehan et al. (2005) Mol Cell Proteomics 4, 346-365. Briefly, lysates are printed on glass backed nitrocellulose array slides (FAST Slides Whatman, Florham Park, N.J.) using a GMS 417 arrayer (Affymetrix, Santa Clara, Calif.) equipped with 500 μm pins, or an Aushon Biosystems 2470 arrayer equipped with 85 μm-350 μm pins. Each lysate is also printed with a set of calibrants as described herein. High and Low controls are also printed for QA/QC assurance. The slides are stored with desiccant (Drierite, W.A. Hammond, Xenia, Ohio) at ±20° C. prior to immunostaining.
Protein Microarray Immunostaining: Immunostaining is performed on an automated slide stainer per manufacturer's instructions (Autostainer CSA kit, Dako, Carpinteria, Calif.). Each slide is incubated with a single primary antibody at room temperature for 30-120 minutes. Suitable primary antibodies will be evident to a skilled worker. For example, polyclonal primary antibodies that can be used to detect the following targets are, e.g.: 14-3-3 zeta/gamma/eta, COX-2, Shc Y317 (Upstate-Millipore; NJ, USA), APC2 (LabVision; Fremont, Calif.), EGFR Y1173, EGFR Y1148 (Invitrogen-Biosource, Carlsbad, Calif.), BUB3, CyclinD1, Cyclin E (BD, Franklin Lakes, N.J.), Beta Actin, 4EBP1 Thr37, BCL-2ser70, EGFR, EGFR Y845, EGFR Y992, EGFR Y1045, EGFR Y1068, EGFR L858R, FKHR Thr24, IRS-1 ser612, mTOR ser2481, ERK T202/Y204, Akt ser473, Akt Thr308, SMAD2 ser465, STAT3 ser727, Src Y416, and Src Y527 (Cell Signaling Technology, Danvers, Mass.). A negative control slide is incubated with antibody diluent. Secondary antibody can be, e.g., goat anti-rabbit IgG H+L (1:5000) (Vector Labs, Burlingame, Calif.).
Bioinformatics method for microarray analysis. Each array is subjected to excitation-emission florescent laser scanning (e.g. LS Reloaded Laser Scanner, TECAN, Durham N.C.) nlaser, and a value for each spot is obtained by subtracting the raw florescent intensity value obtained for each printed spot region on a slide that is stained with just the secondary antibody alone. The values for each spot region that is part of the set of calibrants (calibration curve) are averaged (for each replicate point) and plotted and subjected to non-parametric curve fitting programs (e.g. Curve Fitting Toolbox™, The MathWorks, Natick, Mass.). The intensity values for each background subtracted spot are averaged between replicate printings and the values extrapolated to the set of calibrants (calibration curve), a relative or absolute concentration determined and that value then normalized to total protein obtained by dividing that value by a total protein value that is generated by staining a separate slide with a dye that binds total protein printed without bias (e.g. Sypro Ruby Blot Stain, Molecular Probes, Eugene Oreg.). The total protein value for any given experimental sample is likewise obtained by comparison of the spot region values to a total set of total (not necessarily post-translationally modified) calibrants (protein calibration curve) that is simultaneously printed on the same slide. Such a total protein calibration curve can be generated by printing a dilution curve of known standard proteins such as bovine serum albumin (BSA), or a complex mixture with known stable protein amounts that can be independently measured such as human serum. The intensity values obtained for each analyte and for each sample can then be compared to each other or to clinical data, or other appropriate parameters and analyzed by any standard univariate, and/or multivariate, and/or unsupervised, and/or supervised bioinformatics analysis.

### Example II

### An Illustrative Assay of 26 Analytes

In the present Example, tissue samples from 2 to about 500 patients are prepared and lysed, and aliquots of the whole cell lysates are printed onto slides in a microarray, as described in Example I. Each lysate is printed as a neat and as a 1:4 dilution, each in triplicate. The lysates are printed onto each of 27 slides, each one for the determination of a different analyte in the sample, as well as one slide that is stained for total protein (e.g Sypro Ruby Blot Stain, Molecular Probes Eugene, Oreg.). The analytes to be analyzed/quantitated are:

### 1. Total EGFR

2. Total c-erbB2

### 3. Total VEGFR2 (KDR, Flk-2)

### 4. Total PDGFRbeta

### 5. Total PDGFRalpha

### 6. Total FLT3

### 7. Phospho FLT-3 (Y5899/Y591)

### 8. Phospho VEGFR2 (Y1212)

### 9. Phospho VEGFR1 (Y1213)

10. Phospho PDGFR beta (Y751)/Y735)
11. Phospho PDGFR alpha (Y754)

### 12. Phospho RET (Y905)

### 13. Phospho Src (Y416)

### 14. Phospho AKT (S473)

### 15. Phospho Shc (Y317)

### 16. Phospho Ckit (Y719)

17. Phospho cabl (Y735)
18. Phospho cabl (Y245)
19. Phospho cabl (Y412)

### 20. PhosphoErk (Y42/44)

### 21. Phospho EGFR (Y1086)

### 22. Phospho EGFR (Y1173)

### 23. Phosho EGFR (Y992)

24. Phospho mTOR (S2448)
25. Phospho mTOR (S2481)
26. Phospho p70S6 (T389)

To generate sets of calibrants to measure this collection of analytes, several combinations of cell lines and stimulatory agents are incubated and lysed, and the resulting “upper” and “lower” calibrants are mixed in various combinations to generate three different sets of calibrants. More specifically, the cell line/agent combinations are:

i) HeLa cells unstimulated

ii) HeLa cells stimulated with pervanadate for 5-240 minutes

iii) Jurkat cells stimulated with calyculin for 5-240 minutes

iv) A431 cells stimulated with epidermal growth factor for 5-240 minutes

The analytes produced in these cells are summarized in Table 5 (HeLa unstimulated), Table 3 (HeLa+pervanadate), Table 2 (Jurkat+calyculin), and Table 4 (A431+EGF), respectively.

The cells are lysed, and the volumes of i, ii, iii and iv are adjusted as necessary such that the total amount of protein in each is equivalent. For example, if the total protein content of unstimulated HeLa cells is 5 mg/ml and the protein concentration of HeLa cells stimulated with pervanadate for 30 minutes was 8 mg/ml, as determined by Bradford protein assay, then the stimulated cell lysate would be diluted in the lysing buffer used (e.g. 2.5% beta-mercaptoethanol in T-PER (Pierce, Rockford, Ill.) mixed 1:1 with 2×SDS Tris-glycine buffer (Invitrogen, Carlsbad, Calif.) such that the final concentration is 5 mg/ml.

The adjusted lysate of (ii) is upper calibrator 1; the adjusted lysate of (iii) is upper calibrator 2; and the adjusted lysate of (iv) is upper calibrator 3. Each of these upper calibrators is mixed, independently, with a series of volumes of the adjusted lysate of (i), which is the lower calibrator, to generate a series of intermediate calibrants, having predefined, predictable amounts of the phosphoprotein analytes shown in the Tables, but having a constant amount of total protein. The upper, lower and intermediate calibrants from each of the three mixtures are printed as separate spots, to form sets of calibrants. Each of the three sets of calibrants is printed on each of the 27 slides. Thus, the value of any of the 26 specific analytes can be measured by extrapolation to a curve-fit of one of the 3 sets of calibrants.

The neat spots of the test sample are chosen as a default primary analysis point, and analyzed. If the neat undiluted spots are found to be in saturation when extrapolating to the calibration curve, then the analysis defaults to analysis of the 1:4 dilution spot and the final extrapolated intensity value multiplied by 4 for the dilution factor.

RPMA assays are carried out for each of the 26 analytes as described in Example I.

After background subtraction and secondary antibody alone subtraction, normalization to the total protein slide (e.g., Sypro Ruby Blot stained total protein slide), the intensity values of the neat spot (or the diluted spot, if necessary) are averaged across the replicates and the intensity value is compared against a non-parametrically determined curve generated by the three reference sets of calibrants. A simple extrapolation calculation is used to determine the concentration of the analyte.

### Example III

### Quantitative Analysis of the Total Amount of an Unmodified Protein, c-erbB2

Whole cell lysates of tissue samples from patients having metastatic breast cancer are analyzed as above, except a single set of calibrants is used. To prepare the set of calibrants,

a) A lysate is prepared of SKBR-3 cells (known to have approx 2,390,000±130,000 c-erbB2 receptors/cell, corresponding to a 3+ immunohistochemical score). This is the “upper” calibrant.
b) A lysate is prepared of MDA231 cells (known to have approx 21,600±6700 c-erbB2 receptors/cell, corresponding to a 0 immunohistochemical score). This is the “lower” calibrant.
c) The volumes of either a) or b) are adjusted such that the total amount of protein in each of these calibrants is equivalent.
d) The contents of a) and b) are mixed into predefined, predictable series of dilutions such that the values of c-erbB2 range from the SKBR-3 (upper) value to the MDA 231 (lower) value.

From the foregoing description, one skilled in the art can easily ascertain the essential characteristics of this invention, and without departing from the spirit and scope thereof, can make changes and modifications of the invention to adapt it to various usage and conditions and to utilize the present invention to its fullest extent. The preceding preferred specific embodiments are to be construed as merely illustrative, and not limiting of the scope of the invention in any way whatsoever. The entire disclosure of all applications, patents, and publications cited above, including U.S. Provisional Application Ser. No. 60/970,325, filed Sep. 2, 2007 and U.S. Provisional Application Ser. No. 61/071,324, filed Apr. 22, 2008, and in the figures are hereby incorporated in their entirety by reference.

1.-56. (canceled)

57. A set of calibrants for determining the amount of an analyte in a sample, comprising a plurality of calibrants, which contain a range of amounts of the analyte, spanning the expected amount of the analyte in the sample, wherein, in each of the calibrants, a defined amount of the analyte is present in the same, suitable, biological diluent, wherein the diluent comprises a cell or tissue lysate and the analyte in the lysate is produced by the cell or tissue.

58. The set of calibrants of claim 57, for determining the amount in the sample of at least three analytes, comprising a plurality of calibrants, which contain a range of amounts of each of the at least three analytes, spanning the expected amount of each of the analytes in the sample.

59. The set of calibrants of claim 57, wherein the analyte comprises a non-translationally modified protein.

60. The set of calibrants of claim 59, wherein the analyte comprises erb-B2, c-erbB3, estrogen receptor, androgen receptor, progesterone receptor, EGFR, VEGFR (KDR, Flk-2), c-met, PDGFR, PDGRα, PDGRβ, FLT3, or COX-2.

61. The set of calibrants of claim 57, wherein the analyte comprises a post-translationally modified protein.

62. The set of calibrants of claim 57, wherein the analyte comprises a phosphoprotein isoform.

63. The set of calibrants of claim 57, wherein the analyte comprises one of the proteins listed in Table 1.

64. The set of calibrants of claim 57, wherein
(a) the calibrants are generated by
(i) incubating cells of a suitable cell line with a suitable agent, under conditions such that a high level of the analyte is produced in the cells, and lysing the cells to generate an upper calibrant;
(ii) incubating cells of the cell line of (i) in the absence of the agent, or incubating, in the presence of the agent, cells of a cell line that are not stimulated by the agent or that are stimulated to a low level, and lysing the cells to generate a lower calibrant; and
(iii) mixing, in a series of defined ratios, the upper and lower calibrants of (i) and (ii), to generate a series of calibrants containing intermediate amounts of the analyte, and
(b) the upper, lower and intermediate calibrants are immobilized on a surface which contains, or will contain, samples to be analyzed.

65. The set of calibrants of claim 57, wherein
(a) calibrants for the analyte are generated by
(i) incubating cells of a first cell line, which produce high amounts of the analyte, and lysing the cells to generate an upper calibrant;
(ii) incubating cells of a second cell line, which produce low levels or undetectable amounts of the analyte, and lysing the cells to generate a lower calibrant; and
(iii) mixing, in a series of defined ratios, the upper and lower calibrants of (i) and (ii), to generate a series of calibrants containing intermediate amounts of the analyte, and
(b) the upper, lower and intermediate calibrants are immobilized on a surface which contains, or will contain, samples to be analyzed.

66. The set of calibrants of claim 65, for determining the amount of c-erbB2, wherein calibrants for the c-erbB2 are generated by
(i) incubating cells of the breast cancer line, SKBR3, which overproduces c-erbB2, and lysing the cells to generate an upper calibrant;
(ii) incubating cells of the breast cancer cell line, MDA-231, which under expresses c-erbB2, and lysing the cells to generate a lower calibrant; and
(iii) mixing, in a series of defined ratios, the upper and lower calibrants of (i) and (ii), to generate a series of calibrants containing intermediate amounts of c-erbB2.

67. The set of calibrants of claim 57, which can be used to quantitate at least about 10 analytes, and which comprises a set of at least about 10 calibrants, one for each of the analytes.

68. The set of calibrants of claim 57, which can be used to quantitate at least about 20 analytes, and which comprises a set of at least about 20 calibrants, one for each of the analytes.

69. The set of calibrants of claim 57, which can be used to quantitate at least about 40 analytes, and which comprises a set of at least about 40 calibrants, one for each of the analytes.

70. The set of calibrants of claim 57, which can be used to quantitate at least about 60 analytes, and which comprises a set of at least about 60 calibrants, one for each of the analytes.

71. The set of calibrants of claim 64, wherein the calibrants comprise at least two phosphoprotein isoforms; the suitable cell line is Jurkat; and the suitable agent is FasL or Etoposide.

72. The set of calibrants of claim 64, wherein the calibrants comprise at least 15 phosphoprotein isoforms; the suitable cell line is A431; and the suitable agent is EGF.

73. The set of calibrants of claim 64, wherein the calibrants comprise at least 25 phosphoprotein isoforms, the suitable cell line is HeLa; and the suitable agent is Pervanadate.

74. The set of calibrants of claim 64, wherein the calibrants comprise at least 65 phosphoprotein isoforms; the suitable cell line is Jurkat; and the suitable agent is Calyculin.

75. The set of calibrants of claim 57, which comprises at least about 5 calibrants, each containing a different amount of the analyte.

76. The set of calibrants of claim 57, wherein the amounts of the analyte in the lowest to the highest calibrant span a range of at least 2 orders of magnitude.

77. The set of calibrants of claim 57, wherein the amounts of the analytes in the lowest to the highest calibrant span a range of at least about 5 orders of magnitude.

78. The set of calibrants of claim 57 wherein the sample is obtained from a human.

79. A method for detecting the amount of an analyte in a sample from a subject, comprising
a) immobilizing on a surface an aliquot of the sample and the set of calibrants of claim 57;
b) contacting the sample aliquot and the calibrants of the set of calibrants with a primary antibody that is specific for the analyte, under conditions effective for the primary antibody to specifically interact with the analyte;
c) detecting the interaction of analyte in the sample aliquot and in the calibrants of the set of calibrants with the primary antibody, using a secondary antibody that is specific for the primary antibody, thereby generating a detectable signal that is proportional to the amount of the analyte in the sample aliquot and in the calibrants;
d) comparing the detectable signal obtained from the aliquot to the detectable signals of the series of corresponding calibrants in the set of calibrants; and, optionally,
e) interpolating the amount of signal from the analyte in the sample to a non-parametrically or parametrically determined curve fit of the detectable signals of the calibrants in the set of calibrants, thereby determining the concentration of the analyte in the sample.

80. The method of claim 79, which is a method for detecting the amount of each of at least 3 analytes in a sample from a subject, comprising
a) immobilizing on each of at least 3 separate surfaces an aliquot of the sample, wherein each of the at least 3 surfaces is designated for detecting the amount of one of the at least 3 analytes, and
b) immobilizing on each of the at least 3 surfaces the set of calibrants of claim 57;
c) contacting the sample aliquots and the calibrants of the set of calibrants on each of the at least 3 surfaces with a primary antibody that is specific for the analyte to be detected on that surface, under conditions effective for the primary antibody to specifically interact with the analyte;
d) detecting the interaction of analytes in the sample aliquot and in calibrants of the set of calibrants with the primary antibodies, using a secondary antibody that is specific for the primary antibodies and which is labeled with a detectable moiety that has a dynamic range of at least two orders of magnitude, thereby generating detectable signals that are proportional to the amounts of the analytes in the sample aliquots and in the calibrants;
e) comparing the detectable signal obtained from each aliquot to the detectable signals of the series of corresponding calibrants in the set of calibrants; and, optionally,
f) interpolating the amount of signal from each analyte in the sample to a non-parametrically or parametrically determined curve fit of the detectable signals of the calibrants in the set of calibrants, thereby determining the concentration of the analytes in the sample.

81. The method of claim 79, wherein on each surface, at least one undiluted (neat) aliquot of the sample and at least one diluted aliquot of the sample are immobilized on the surface, and each sample aliquot is present in a sufficient number of replicates for statistical robustness.

82. The method of claim 80, wherein on each surface, at least one undiluted (neat) aliquot of the sample and at least one diluted aliquot of the sample are immobilized on the surface, and each sample aliquot is present in a sufficient number of replicates for statistical robustness.

83. The method of claim 79, wherein the analyte is a phosphoprotein isoform that forms part of one or more kinase signaling pathways.

84. The method of claim 79, wherein the analyte comprises one of the phosphoproteins listed in Tables 2, 3, 4 and/or 5.

85. The method of claim 79, wherein the analyte comprises c-erbB2 (Y1248) or EGFR (Y845, Y1045, Y1068, Y1148, or Y1173).

86. The method of claim 79, wherein the analyte comprises one of the proteins listed in Table 1.

87. The method of claim 79, wherein the analyte comprises c-erbB2, and the set of calibrants is generated by
(i) incubating cells of the breast cancer line, SKBR3, which overproduces c-erbB2, and lysing the cells to generate an upper calibrant;
(ii) incubating cells of the breast cancer cell line, MDA-231, which under expresses c-erbB2, and lysing the cells to generate a lower calibrant; and
(iii) mixing, in a series of defined ratios, the upper and lower calibrants of (i) and (ii), to generate a series of calibrants containing intermediate amounts of c-erbB2.

88. The method of method of claim 79, wherein the amounts of at least five analytes are detected for each sample.

89. The method of method of claim 79, wherein aliquots of at least about 200 samples are immobilized on the surface and are analyzed to determine the amounts of the analytes in the samples.

90. The method claim 79, wherein the samples are analyzed by RPMA (reverse phase protein microarray) analysis.

91. The method claim 90 wherein the secondary antibody is labeled with a dye that has a dynamic range of at least two orders of magnitude, and wherein the calibrants in the set of calibrants span a concentration range of at least two orders of magnitude.

92. The method of claim 79, wherein the subject is a human.

93. A kit for performing a calibrated RPMA assay, comprising
a) the set of calibrants of claim 57, and
b) suitable reagents.

94. The kit of claim 93 wherein the calibrants are stored as frozen liquids or as lyophilized samples.

95. The kit of claim 93 wherein the set of calibrants of claim 57 are immobilized on a surface.

96. The kit of claim 95 wherein the calibrants are stored as frozen liquids or as lyophilized samples.

97. A method for making a set of calibrants for detecting an analyte of interest, comprising
a) incubating cells of a suitable cell line with a suitable agent, under conditions such that a high level of the analyte is produced in the cells, and lysing the cells to generate an upper calibrant;
b) incubating the cell line of a) in the absence of the agent, or incubating, in the presence of the agent, a cell line that is not stimulated by the agent or that is stimulated to a low level, and lysing the cells to generate a lower calibrant; and
c) mixing, in a series of defined ratios, the upper and lower calibrants of a) and b), to generate a series of calibrants containing intermediate amounts of the analyte, and
d) immobilizing the upper, lower and intermediate calibrants on a surface.

98. A surface onto which is immobilized the set of calibrants of claim 57.

99. The surface of claim 98, wherein the surface is nitrocellulose.
